(19) United States
(12) Patent Application Publication
(10) Pub. No.: US 2002/0018776 A1
(43) Pub. Date: Feb. 14, 2002

(54) Method of treating graft rejection using inhibitors of CXCR3 function

(75) Inventor: Wayne W. Hancock, Medfield, MA (US)

Correspondence Address: Helen E. Wendler, Esq., HAMILTON, BROOK, SMITH & REYNOLDS, P.C., Two Militia Drive, Lexington, MA, 02421-4799, US

(73) Assignee: Millennium Pharmaceuticals, Inc., Cambridge, MA (US)

(21) Appl. No.: 09/835,103

(22) Filed: Apr. 13, 2001

Related U.S. Application Data
(63) Continuation-in-part of application No. 09/549,856, filed on Apr. 14, 2000, now abandoned.

(51) Int. Cl. A61K 39/395; A61K 38/13; A61K 31/573; A61K 31/4745

(52) U.S. Cl. 424/145.1; 514/9; 514/179; 514/291

(57) ABSTRACT

A method for inhibiting the rejection of transplanted grafts is disclosed. The method comprising administering an effective amount of an antagonist of CXCR3 function to a graft recipient. The disclosed methods can also comprise the co-administration of one or more additional therapeutic agents, for example, immunosuppressive agents.

### RELATED APPLICATIONS

This application is a continuation-in-part of U.S. application Ser. No. 09/549,856, filed Apr. 14, 2000, the entire teachings of which are incorporated herein by reference.

### BACKGROUND OF THE INVENTION

In many instances, the best and only treatment available to patients suffering from certain end stage degenerative conditions or congenital genetic disorders is transplantation of a healthy graft (e.g., organs, tissues). Advances in surgical techniques and post-operative immunosuppressive therapy have mitigated some of the barriers to long-term survival of grafts and graft recipients, and ushered this once experimental therapy into wider clinical practice.

A major barrier to the long-term survival of transplanted grafts is rejection by the recipient's immune system. Graft rejection can be classified as hyper-acute rejection which is mediated by preformed antibodies that can bind to the graft and are present in the circulation of the recipient, acute rejection which is mediated by the recipient's cellular immune response or chronic rejection which occurs via a multi-factorial process that includes an immune component. The practice of matching the allelic variants of cellular antigens, most notably major histocompatibility antigens (MHC), also referred to as tissue typing, as well as matching of the blood type of the donor and recipient has reduced the incidence of hyper-acute rejection. However, most grafts which are transplanted do not exactly match the tissue type of the recipient (e.g., allografts) and will not remain viable without therapeutic intervention.

The rejection of allografts can be inhibited by long-term (e.g., life-long) prophylactic immunosuppressive therapy, most notably with agents that inhibit calcineurin (e.g., cyclosporin A (CsA), FK-506). Immunosuppressive therapy not only inhibits rejection of the graft, but can render the recipient susceptible to infection with, for example, viruses, bacteria and fungi (e.g., yeasts, molds), and at higher risk for the development of certain malignancies. Additionally, therapeutic doses of immunosuppressive agents can produce adverse side effects, such as diabetes mellitus, neurotoxicity, nephrotoxicity, hyperlipidemia, hypertension, hirsutism and gingival hyperplasia (Spencer, C. M., et al., Drugs 54(6):925-975 (1997)). Thus, the degree of immunosuppression must be carefully tailored to prevent rejection of the graft and to preserve the general health of the recipient.

Despite such prophylactic immunosuppression, acute and chronic rejection of grafts remains a clinical problem. Acute episodes of rejection are characterized by infiltration of the graft by the recipient's leukocytes (e.g., monocytes, macrophages, T cells) and cellular necrosis. These episodes usually occur during the days to months following transplantation. Acute rejection has been treated with high doses of certain immunosuppressive agents, such as glucocorticoids (e.g., prednisone) and certain antibodies which bind to leukocytes (e.g., OKT3). However, these therapies do not always stop the rejection, are associated with systemic side effects and can lose efficacy in cases of recurrent rejection activity.

Chronic rejection becomes the major cause of graft failure and recipient death for those patients that survive past the first year. For example, evidence of chronic rejection can be found in about 40-50% of heart and/or lung allograft recipients who survive for five years, and most kidney grafts succumb to chronic rejection. The pathogenesis of chronic rejection is complex and involves accelerated arteriosclerosis (e.g., atherosclerosis) of the graft-associated vasculature and leukocyte infiltration. Unlike acute rejection episodes, chronic rejection is not generally responsive to further immunosuppressive therapy. Furthermore, the graft accelerated arteriosclerosis characteristic of chronic rejection is generally diffuse and not amenable to conventional therapeutic procedures (e.g., angioplasty, bypass grafting, endarterectomy). Thus, patients who chronically reject their grafts can require a second transplant (Schroeder J. S. “Cardiac Transplantation”, pp. 1298-1300; Maurer, J. R. “Lung Transplantation”, pp. 1491-1493; Carpenter, C. B. and Lazarus, J. M. “Dialysis and Transplantation in the Treatment of Renal Failure”, pp. 1524-1529; Dienstag, J. “Liver Transplantation”, pp. 1721-1725; all in Harrison's Principles of Internal Medicine, 14^{th} ed., Fauci et al. Eds. McGraw Hill (1998)).

A need exists for therapeutic methods for preventing graft rejection.

### SUMMARY OF THE INVENTION

The invention relates to transplantation and to promoting the viability of transplanted grafts. In one aspect, the invention relates to a method for inhibiting (reducing or preventing) graft rejection (e.g., acute rejection, chronic rejection). In one embodiment, the method comprises administering to a graft recipient an effective amount of an antagonist of CXCR3 function. In another embodiment, the graft is an allograft. In a particular embodiment, the allograft is a heart. In a preferred embodiment, the method comprises administration of an effective amount of an antagonist of CXCR3 function and an effective amount of one or more immunosuppressive agents to a graft recipient.

The invention also relates to a method for diagnosing graft rejection. In one embodiment, the method comprises assessing (detecting or measuring) the expression of IP-10 by the graft. In another embodiment, the method comprises assessing (detecting or measuring) graft infiltration by CXCR3⁺ cells.

### BRIEF DESCRIPTION OF THE DRAWINGS

FIG. 1 is a histogram illustrating concanavalin-A (Con-A) induced proliferation of T cells in cultures of splenocytes isolated from CXCR3 KO (CXCR3−/− on C57BL/6 background) or WT (wild type, CXCR3+/+ on C57BL/6 background) mice. T cells from CXCR3 KO or WT mice were cultured in media that contained various concentrations of concanavalin-A (Con-A; 0, 1.25, 2.5, 5 or 10 μg/ml) for 72 hours. The induced T cell proliferative responses in cultures of cells from CXCR3 KO mice or cultures of cells from CXCR3+/+ mice were about equivalent. Data are mean±standard deviation of twelve cultures.

FIG. 2 is a histogram illustrating mixed lymphocyte responses (MLRs) of cells isolated from CXCR3 KO (CXCR3−/− on C57BL/6 background; KO) or WT (wild type, CXCR3+/+ on C57BL/6 background) mice stimulated with allogeneic splenocytes isolated from BALB/c mice. Cells from CXCR3−/− (KO) or CXCR3+/+ (WT) mice displayed robust MLRs toward mitomycin-C-treated BALB/c stimulator cells. However, CXCR3−/− (KO) cells proliferated less than wild type (WT) cells in the assay (*p<0.001, Mann-Whitney U test). Data are mean±standard deviation of twelve three- to five-day cultures. BALB alone: mitomycin-C-treated splenocytes isolated from BALB/c mice; WT alone: splenocytes isolated from CXCR3+/+ (C57BL/6) mice; KO alone: splenocytes isolated from CXCR3−/− (C57BL/6) mice; BALB→WT: splenocytes isolated from CXCR3+/+ (C57BL/6) mice stimulated with mitomycin-C-treated splenocytes isolated from BALB/c mice; BALB→KO: splenocytes isolated from CXCR3−/− (C57BL/6) mice stimulated with mitomycin-C-treated splenocytes isolated from BALB/c mice.

FIG. 3 is a histogram illustrating dose-dependent inhibition by anti-CXCR3 mAb 4C4 (mAb) of mixed lymphocyte responses (MLRs) of cells isolated from WT (wild type, C57BL/6) mice and stimulated with allogeneic splenocytes isolated from BALB/c mice. MLRs were significantly inhibited in cultures that contained anti-CXCR3 mAb 4C4 (mAb), but not in cultures that contained an IgM control antibody (IgM) (asterisks indicate significantly decreased proliferation of cells in the presence of CXCR3 mAb 4C4 (mAb) as compared to proliferation of cells in the presence of an IgM control antibody; *p<0.005, **p<0.001, Mann-Whitney U test). Data are mean±standard deviation of twelve cultures maintained for three to five days. Anti-CXCR3 mAb 4C4 (mAb) and IgM control antibody (IgM) concentrations were 10, 1 or 0.1 μg/ml, as indicated. BALB/c stimulators: mitomycin-C-treated splenocytes isolated from BALB/c mice; WT responders: splenocytes isolated from WT (C57BL/6) mice; BALB→WT/IgM 10: splenocytes isolated from WT (C57BL/6) mice stimulated with mitomycin-C-treated splenocytes isolated from BALB/c mice, the cultures contained IgM control antibody (10 μg/ml); BALB→WT/IgM 1: splenocytes isolated from WT (C57BL/6) mice stimulated with mitomycin-C-treated splenocytes isolated from BALB/c mice, the cultures contained IgM control antibody (1 μg/ml); BALB-WT/IgM 0.1: splenocytes isolated from WT (C57BL/6) mice stimulated with mitomycin-C-treated splenocytes isolated from BALB/c mice, the cultures contained IgM control antibody (0.1 μg/ml); BALB→WT/mAb 10: splenocytes isolated from WT (C57BL/6) mice stimulated with mitomycin-C-treated splenocytes isolated from BALB/c mice, the cultures contained anti-CXCR3 mAb 4C4 (10 μg/ml); BALB→WT/mAb 1: splenocytes isolated from WT (C57BL/6) mice stimulated with mitomycin-C-treated splenocytes isolated from BALB/c mice, the cultures contained anti-CXCR3 mAb 4C4 (1 μg/ml); BALB→WT/mAb 0.1: splenocytes isolated from WT (C57BL/6) mice stimulated with mitomycin-C-treated splenocytes isolated from BALB/c mice, the cultures contained anti-CXCR3 mAb 4C4 (0.1 μg/ml).

FIG. 4 is a histogram illustrating that anti-CXCR3 mAb 4C4 (mAb) prolongs cardiac allograft survival of BALB/c allografts transplanted into WT (C57BL/6) recipients. Administration of anti-CXCR3 mAb 4C4 (mAb) (500 μg by intraperitoneal injection every 48 hours until day 14) prolonged the survival of allogeneic cardiac allografts when administration began at transplantation (day 0, d 0). The survival of allogeneic cardiac allografts was also prolonged when anti-CXCR3 mAb 4C4 (mAb) administration was initiated after rejection had begun (day 4, d +4). Data are mean±standard deviation for 6 mice. Asterisks indicate significantly increased prolongation of allograft survival in recipients that were administered anti-CXCR3 mAb 4C4 (mAb) as compared with recipients that were administered an IgM control antibody (IgM) (p<0.001, Mann-Whitney U test).

### DETAILED DESCRIPTION OF THE INVENTION

The invention relates to transplantation and to promoting the viability of transplanted grafts. Specifically, the invention relates to inhibiting graft rejection (e.g., acute graft rejection, chronic graft rejection) by administering to a graft recipient an effective amount of an antagonist of mammalian (e.g., human, Homo sapiens) CXC chemokine receptor 3, CXCR3.

Chemokines are a family of proinflammatory mediators that promote recruitment and activation of multiple lineages of leukocytes (e.g., lymphocytes, macrophages). They can be released by many kinds of tissue cells after activation. Continuous release of chemokines at sites of inflammation can mediate the ongoing migration and recruitment of effector cells to sites of chronic inflammation. The chemokines are related in primary structure and share four conserved cysteines, which form disulfide bonds. Based upon this conserved cysteine motif, the family can be divided into distinct branches, including the C-X-C chemokines (α-chemokines), and the C-C chemokines (β-chemokines), in which the first two conserved cysteines are separated by an intervening residue, or are adjacent residues, respectively (Baggiolini, M. and Dahinden, C. A., Immunology Today, 15:127-133 (1994)).

The C-X-C chemokines include a number of potent chemoattractants and activators of neutrophils, such as interleukin 8 (IL-8), PF4 and neutrophil-activating peptide-2 (NAP-2). The C-C chemokines include, for example, RANTES (Regulated on Activation, Normal T Expressed and Secreted), macrophage inflammatory proteins 1α and 1β (MIP-1α and MIP-1β), eotaxin and human monocyte chemotactic proteins 1-3 (MCP-1, MCP-2, MCP-3), which have been characterized as chemoattractants and activators of monocytes or lymphocytes. Chemokines, such as IL-8, RANTES and MIP-1α, for example, have been implicated in human acute and chronic inflammatory diseases including respiratory diseases, such as asthma and allergic disorders.

The chemokine receptors are members of a superfamily of G protein-coupled receptors (GPCR) which share structural features that reflect a common mechanism of action of signal transduction (Gerard, C. and Gerard, N. P., Annu Rev. Immunol., 12:775-808 (1994); Gerard, C. and Gerard, N. P., Curr. Opin. Immunol., 6:140-145 (1994)). Conserved features include seven hydrophobic domains spanning the plasma membrane, which are connected by hydrophilic extracellular and intracellular loops. The majority of the primary sequence homology occurs in the hydrophobic transmembrane regions with the hydrophilic regions being more diverse. The receptors for the C-C chemokines include: CCR1 which can bind, for example, MIP-1α, RANTES, MCP-2, MCP-3, MCP-4, CKbeta8, CKbeta8-1, leukotactin-1, HCC-1 and MPIF-1; CCR2 which can bind, for example, MCP-1, MCP-2, MCP-3 and MCP-4; CCR3 which can bind, for example, eotaxin, eotaxin-2, RANTES, MCP-2, MCP-3 and MCP-4; CCR4 which can bind, for example, TARC, RANTES, MIP-1α and MCP-1; CCR5 which can bind, for example, MIP-1α, RANTES, and MIP-1β; CCR6 which can bind, for example, LARC/MIP-3α/exodus; CCR7 which can bind, for example, ELC/MIP-3β; CCR8 which can bind, for example, 1-309; CCR9 which can bind, for example, TECK and CCR10 which can bind, for example, ESkine and CCL27 (Baggiolini, M., Nature 392:565-568 (1998); Luster, A. D., New England Journal of Medicine, 338(7):436-445 (1998); Tsou, et al., J. Exp. Med., 188:603-608 (1998); Nardelli, et al., J Immunol, 162(1):435-444 (1999); Youn, et al., Blood, 91(9):3118-3126 (1998); Youn, et al., J Immunol, 159(11):5201-5205 (1997); Zaballos, et al., J Immunol, 162:5671-5675 (1999); Jarmin, et al., J Immunol, 164:3460-3464 (2000); Homey et al., J Immunol, 164:3465-3470 (2000)). The receptors for the CXC chemokines include: CXCR1 which can bind, for example, IL-8, GCP-2; CXCR2 which can bind, for example, IL-8, GROα/β/γ, NAP-2, ENA78, GCP-2; CXCR3 which can bind, for example, interferon gamma (IFNγ)-inducible protein of 10 kDa (IP-10), monokine induced by IFNγ (Mig), interferon-inducible T cell chemoattractant (1-TAC); CXCR4 which can bind, for example, SDF-1; and CXCR5 which can bind, for example, BCA-1/BLC (Baggiolini M., Nature, 392:565-568 (1998); Lu et al., Eur J Immunol, 29:3804-3812 (1999)).

CXCR3, as well as processes and cellular responses mediated by CXCR3, are involved in rejection of transplanted grafts. As described herein, studies of allograft survival using a murine cardiac transplantation model were undertaken. Mice which lacked functional chemokine receptor CXCR3 as a result of targeted disruption of the CXCR3 gene (CXCR3 KO mice) did not reject transplanted allografts, which were mismatched at MHC class I and MHC class II, as rapidly as control mice which had a functional CXCR3 gene (CXCR3+/+) and were otherwise genetically identical to CXCR3 KO mice (Example 1, Table 1, groups 1 and 2, 6 and 7).

As described herein, administration of low dose immunosuppressive therapy (cyclosporin A (CsA)) to CXCR3+/+ control mice resulted in about a 3-day increase in graft viability as compared with untreated CXCR3+/+ animals (Example 1, Table 2, group 12). Surprisingly, administration of the same low dose of CsA with inhibition of CXCR3 function led to permanent (>100 days) engraftment in CXCR3 KO mice which received CsA for a maximum period of only 14 days (Example 1, Table 2, group 14).

Histological examination of permanently engrafted hearts removed from group 14 mice (see Table 2) at 100 days after transplantation revealed only minimal mononuclear cell infiltration and no evidence of transplant accelerated arteriosclerosis.

The survival of Class I and Class II mismatched allografts can be prolonged by the administration of anti-CD4 monoclonal antibody (mAb) (Mottram, et al., Transplantation, 59:559-565 (1995)). However, the long-term survival of these grafts is complicated by the development of chronic rejection with wide-spread arteriosclerosis in the vasculature of the graft (Hancock, et al., Nature Medicine, 4:1392-1396 (1998)). As described herein, Class I and Class II mismatched allografts survived for at least 60 days in CXCR3+/+ control mice that received anti-CD4 mAb therapy and for at least 100 days in CXCR3 KO mice that were treated with low dose CsA for 14 days following transplantation. Morphological examination of grafts which were removed from CXCR3+/+ control recipients that received anti-CD4 therapy after about sixty days, revealed severe arteriosclerosis. In contrast, grafts which were removed from CXCR3 KO recipients treated with low dose CsA, about 100 days after transplantation, showed no evidence of arteriosclerosis by morphological examination (Example 2, Table 3). Thus, disruption of CXCR3 function can provide the dual benefit of inhibiting both acute and chronic rejection of allografts.

Accordingly, a first aspect of the invention provides a method for inhibiting rejection (e.g., acute and/or chronic rejection) of a graft, comprising administering to a graft recipient an effective amount of an antagonist of CXCR3 function.

### CXCR3 antagonists

As used herein, the term “antagonist of CXCR3 function” refers to an agent (e.g., a molecule, a compound) which can inhibit a (i.e., one or more) function of CXCR3. For example, an antagonist of CXCR3 function can inhibit the binding of one or more ligands (e.g., IP-10, Mig, I-TAC) to CXCR3 and/or inhibit signal transduction mediated through CXCR3 (e.g., GDP/GTP exchange by CXCR3-associated G proteins, intracellular calcium flux). Accordingly, CXCR3-mediated processes and cellular responses (e.g., proliferation, migration, chemotactic responses, secretion or degranulation) can be inhibited with an antagonist of CXCR3 function. As used herein, “CXCR3” refers to naturally occurring CXC chemokine receptor 3 (e.g., mammalian CXCR3 (e.g., human (Homo sapiens) CXCR3)) and encompasses naturally occurring variants, such as allelic variants and splice variants (see, for example, WO 98/11218 and WO 99/50299).

Preferably, the antagonist of CXCR3 function is a compound which is, for example, a small organic molecule, natural product, protein (e.g., antibody, chemokine, cytokine), peptide or peptidomimetic. Several molecules that can antagonize one or more functions of chemokine receptors (e.g., CXCR3) are known in the art, including the small organic molecules disclosed in, for example, international patent application WO 97/24325 by Takeda Chemical Industries, Ltd.; WO 98/38167 by Pfizer, Inc.; WO 97/44329 by Teijin Limited; WO 98/04554 by Banyu Pharmaceutical Co., Ltd.; WO 98/27815, WO 98/25604, WO 98/25605, WO 98/25617 and WO 98/31364 by Merck & Co., Inc.; Hesselgesser et al., J. Biol. Chem. 273(25):15687-15692 (1998); and Howard et al., J. Medicinal Chem. 41(13):2184-2193 (1998); proteins, such as antibodies (e.g., polyclonal sera, monoclonal, chimeric, humanized, human) and antigen-binding fragments thereof (e.g., Fab, Fab′, F(ab′)₂, Fv), for example, those disclosed in WO 98/11218 by Theodor-Kocher Institute and LeukoSite, Inc.; chemokine mutants and analogues, for example, those disclosed in U.S. Pat. No. 5,739,103 issued to Rollins et al., WO 96/38559 by Dana Farber Cancer Institute and WO 98/06751 by Research Corporation Technologies, Inc.; peptides, for example, those disclosed in WO 98/09642 by The United States of America. The entire teachings of each of the above cited patents, patent applications and references are incorporated herein by reference.

Antagonists of CXCR3 function can be identified, for example, by screening libraries or collections of molecules, such as, the Chemical Repository of the National Cancer Institute, as described herein or using other suitable methods.

Another source of antagonists of CXCR3 function are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods, such as the methods described herein.

The term “natural product”, as used herein, refers to a compound which can be found in nature, for example, naturally occurring metabolites of marine organisms (e.g., tunicates, algae), plants or other organisms, and which possesses biological activity, e.g., can antagonize CXCR3 function. For example, lactacystin, paclitaxel and cyclosporin A are natural products which can be used as anti-proliferative or immunosuppressive agents.

Natural products can be isolated and identified by suitable means. For example, a suitable biological source (e.g., vegetation) can be homogenized (e.g., by grinding) in a suitable buffer and clarified by centrifugation, thereby producing an extract. The resulting extract can be assayed for the capacity to antagonize CXCR3 function, for example, by the assays described herein. Extracts which contain an activity that antagonizes CXCR3 function can be further processed to isolate the CXCR3 antagonist by suitable methods, such as, fractionation (e.g., column chromatography (e.g., ion exchange, reverse phase, affinity), phase partitioning, fractional crystallization) and assaying for biological activity (e.g., antagonism of CXCR3 activity). Once isolated the structure of a natural product can be determined (e.g., by nuclear magnetic resonance (NMR)) and those of skill in the art can devise a synthetic scheme for synthesizing the natural product. Thus, a natural product can be isolated (e.g., substantially purified) from nature or can be fully or partially synthetic. A natural product can be modified (e.g., derivatized) to optimize its therapeutic potential. Thus, the term “natural product”, as used herein, includes those compounds which are produced using standard medicinal chemistry techniques to optimize the therapeutic potential of a compound which can be isolated from nature.

The term “peptide”, as used herein, refers to a compound consisting of from about two to about ninety amino acid residues wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). A “peptide” can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, omithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art and are disclosed in, for example, Green and Wuts, “Protecting Groups in Organic Synthesis”, John Wiley and Sons, 1991. The functional groups of a peptide can also be derivatized (e.g., alkylated) using art-known methods.

Peptides can be synthesized and assembled into libraries comprising a few to many discrete molecular species. Such libraries can be prepared using well-known methods of combinatorial chemistry, and can be screened as described herein or using other suitable methods to determine if the library comprises peptides which can antagonize CXCR3 function. Such peptide antagonists can then be isolated by suitable methods.

The term “peptidomimetic”, as used herein, refers to molecules which are not polypeptides, but which mimic aspects of their structures. For example, polysaccharides can be prepared that have the same functional groups as peptides which can antagonize CXCR3. Peptidomimetics can be designed, for example, by establishing the three dimensional structure of a peptide agent in the environment in which it is bound or will bind to CXCR3. The peptidomimetic comprises at least two components, the binding moiety or moieties and the backbone or supporting structure.

The binding moieties are the chemical atoms or groups which will react or form a complex (e.g., through hydrophobic or ionic interactions) with CXCR3, for example, with the amino acid(s) at or near the ligand binding site. For example, the binding moieties in a peptidomimetic can be the same as those in a peptide antagonist of CXCR3. The binding moieties can be an atom or chemical group which reacts with the receptor in the same or similar manner as the binding moiety in a peptide antagonist of CXCR3. Examples of binding moieties suitable for use in designing a peptidomimetic for a basic amino acid in a peptide are nitrogen containing groups, such as amines, ammoniums, guanidines and amides or phosphoniums. Examples of binding moieties suitable for use in designing a peptidomimetic for an acidic amino acid can be, for example, carboxyl, lower alkyl carboxylic acid ester, sulfonic acid, a lower alkyl sulfonic acid ester or a phosphorous acid or ester thereof.

The supporting structure is the chemical entity that, when bound to the binding moiety or moieties, provides the three dimensional configuration of the peptidomimetic. The supporting structure can be organic or inorganic. Examples of organic supporting structures include polysaccharides, polymers or oligomers of organic synthetic polymers (such as, polyvinyl alcohol or polylactide). It is preferred that the supporting structure possess substantially the same size and dimensions as the peptide backbone or supporting structure. This can be determined by calculating or measuring the size of the atoms and bonds of the peptide and peptidomimetic. In one embodiment, the nitrogen of the peptide bond can be substituted with oxygen or sulfur, thereby forming a polyester backbone. In another embodiment, the carbonyl can be substituted with a sulfonyl group or sulfinyl group, thereby forming a polyamide (e.g., a polysulfonamide). Reverse amides of the peptide can be made (e.g., substituting one or more —CONH— groups for a —NHCO— group). In yet another embodiment, the peptide backbone can be substituted with a polysilane backbone.

These compounds can be manufactured by known methods. For example, a polyester peptidomimetic can be prepared by substituting a hydroxyl group for the corresponding α-amino group on amino acids, thereby preparing a hydroxyacid and sequentially esterifying the hydroxyacids, optionally blocking the basic and acidic side chains to minimize side reactions. An appropriate chemical synthesis route can generally be readily identified upon determining the desired chemical structure of the peptidomimetic.

Peptidomimetics can be synthesized and assembled into libraries comprising a few to many discrete molecular species. Such libraries can be prepared using well-known methods of combinatorial chemistry, and can be screened as described herein to determine if the library comprises one or more peptidomimetics which antagonize CXCR3 function. Such peptidomimetic antagonists can then be isolated by suitable methods.

In one embodiment, the CXCR3 antagonist is an antibody or antigen-binding fragment thereof having specificity for CXCR3. The antibody can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody” as used herein also encompasses functional fragments of antibodies, including fragments of chimeric, human, humanized, primatized, veneered or single-chain antibodies. Functional fragments include antigen-binding fragments which bind to CXCR3. For example, antibody fragments capable of binding to CXCR3 or portions thereof, including, but not limited to Fv, Fab, Fab′ and F(ab′)₂ fragments can be used. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain. Single-chain antibodies, and chimeric, human, humanized or primatized (CDR-grafted), or veneered antibodies, as well as chimeric, CDR-grafted or veneered single-chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989)); Sato, K., et al., Cancer Research, 53: 851-856 (1993); Daugherty, B. L. et al., Nucleic Acids Res., 19(9): 2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

Antibodies which are specific for mammalian (e.g., human) CXCR3 can be raised against an appropriate immunogen, such as isolated and/or recombinant human CXCR3 or portions thereof (including synthetic molecules, such as synthetic peptides). Antibodies can also be raised by immunizing a suitable host (e.g., mouse, rat) with cells that express CXCR3, such as activated T cells (see, e.g., U.S. Pat. No. 5,440,020, the entire teachings of which are incorporated herein by reference). In addition, cells expressing recombinant CXCR3 such as transfected cells, can be used as immunogens or in a screen for antibody which binds receptor (see, e.g., Chuntharapai et al., J. Immunol., 152: 1783-1789 (1994); Chuntharapai et al., U.S. Pat. No. 5,440,021; and WO 98/11218 (Theodor-Kocher Institute et al.), published Mar. 19, 1998).

Preparation of immunizing antigen, and polyclonal and monoclonal antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). When a monoclonal antibody is desired, a hybridoma can generally be produced by fusing a suitable immortal cell line (e.g., a myeloma cell line such as SP2/0 or P3X63Ag8.653) with antibody-producing cells. The antibody-producing cells, preferably those obtained from the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity can be used, including, for example, methods which select recombinant antibody from a library (e.g., a phage display library). Transgenic animals capable of producing a repertoire of human antibodies (e.g., XenoMouse™ (Abgenix, Fremont, Calif.)) can be produced using suitable methods (see, e.g., WO 98/24893 (Abgenix), published Jun. 11, 1998; Kucherlapati, R. and Jakobovits, A., U.S. Pat. No. 5,939,598; Jakobovits et al., Proc. Natl. Acad. Sci. USA, 90: 2551-2555 (1993); Jakobovits et al., Nature, 362: 255-258 (1993)). Additional methods for production of transgenic animals capable of producing a repertoire of human antibodies have been described (e.g., Lonberg et al., U.S. Pat. No. 5,545,806; Surani et al., U.S. Pat. No. 5,545,807; Lonberg et al., WO97/13852).

In one embodiment, the antibody or antigen-binding fragment thereof has specificity for a mammalian CXC chemokine receptor 3 (CXCR3), such as human CXCR3. In a preferred embodiment, the antibody or antigen-binding fragment can inhibit binding of a ligand (i.e., one or more ligands) to CXCR3 and/or one or more functions mediated by CXCR3 in response to ligand binding. Preferred antibody antagonists of CXCR3 function, such as murine mAb 1C6 are disclosed in WO 98/11218 and corresponding U.S. application Ser. No. 08/829,839, filed Mar. 31, 1997 (now U.S. Pat. No. 6,184,358), the teachings of both of which are incorporated herein by reference in their entirety. This antibody and, for example, chimeric or humanized versions of this antibody, can be administered in accordance with the invention.

Murine hybridoma 1C6 (also referred to as LS77 1C6-3) which produces murine mAb 1C6 was deposited on Mar. 28, 1997 on behalf of LeukoSite, Inc., 215 First Street, Cambridge, Mass. 02142 (now Millennium Pharmaceuticals, Inc., 75 Sidney Street, Cambridge, Mass. 02139), at the American Type Culture Collection, 10801 University Boulevard, Manassas, Va., 20110, in accordance with the terms of the Budapest Treaty, under Accession Number HB-12330.

Antibodies which bind CXCR3 ligand (e.g., IP-10, Mig, I-TAC) and inhibit binding of ligand to CXCR3 can be prepared using any suitable method, such as the methods described herein.

### Assessment of Activity of Antagonists

The capacity of an agent (e.g., proteins, peptides, natural products, small organic molecules, peptidomimetics) to antagonize CXCR3 function can be determined using a suitable screen (e.g., high through-put assay). For example, an agent can be tested in an extracellular acidification assay, calcium flux assay, ligand binding assay or chemotaxis assay (see, for example, WO 98/11218, published Mar. 19, 1998; Hesselgesser et al., J. Biol. Chem. 273(25):15687-15692 (1998) and WO 98/02151).

In a particular assay, membranes can be prepared from cells which express CXCR3, such as activated T cells or cells which express recombinant CXCR3. Cells can be harvested by centrifugation, washed twice with PBS (phosphate-buffered saline), and the resulting cell pellets frozen at −70 to −85° C. The frozen pellet can be thawed in ice-cold lysis buffer consisting of 5 mM HEPES (N-2-hydroxyethylpiperazine-N′-2-ethane-sulfonic acid) pH 7.5, 2 mM EDTA (ethylenediaminetetraacetic acid), 5 μg/ml each aprotinin, leupeptin, and chymostatin (protease inhibitors), and 100 μg/ml PMSF (phenyl methane sulfonyl fluoride—also a protease inhibitor), at a concentration of 1 to 5×10⁷ cells/ml, to achieve cell lysis. The resulting suspension can be mixed well to resuspend all of the frozen cell pellet. Nuclei and cell debris can be removed by centrifugation at 400×g for 10 minutes at 4° C. The resulting supernatant can be transferred to a fresh tube and the membrane fragments can be collected by centrifugation at 25,000×g for 30 minutes at 4° C. The resulting supernatant can be aspirated and the pellet can be resuspended in freezing buffer consisting of 10 mM HEPES pH 7.5, 300 mM sucrose, 1 μg/ml each aprotinin, leupeptin, and chymostatin, and 10 μg/ml PMSF (approximately 0.1 ml per each 10⁸ cells). All clumps can be resolved using a minihomogenizer, and the total protein concentration can be determined by suitable methods (e.g., Bradford assay, Lowery assay). The membrane solution can be divided into aliquots and frozen at −70 to −85° C. until needed.

The membrane preparation described above can be used in a suitable binding assay. For example, membrane protein (2 to 20 μg total membrane protein) can be incubated with 0.1 to 0.2 nM ¹²⁵I-labeled IP-10, ¹²⁵I-labeled Mig or ¹²⁵I-labeled I-TAC with or without unlabeled competitor (IP-10, Mig, I-TAC) or various concentrations of compounds to be tested. ¹²⁵I-labeled IP-10, ¹²⁵I-labeled Mig and ¹²⁵I-labeled I-TAC can be prepared by suitable methods or purchased from commercial vendors (e.g., DuPont-NEN (Boston, Mass.)). The binding reactions can be performed in 60 to 100 μl of binding buffer consisting of 10 mM HEPES pH 7.2, 1 mM CaCl₂, 5 mM MgCl₂ and 0.5% BSA (bovine serum albumin), for 60 min at room temperature. The binding reactions can be terminated by harvesting the membranes by rapid filtration through glass fiber filters (e.g., GF/B or GF/C, Packard) which can be presoaked in 0.3% polyethyleneimine. The filters can be rinsed with approximately 600 μl of binding buffer containing 0.5 M NaCl, dried, and the amount of bound radioactivity can be determined by scintillation counting.

The CXCR3 antagonist activity of test agents (e.g., compounds) can be reported as the inhibitor concentration required for 50% inhibition (IC₅₀ values) of specific binding in receptor binding assays (e.g., using ¹²⁵I-IP-10, ¹²⁵I-Mig or ¹²⁵I-I-TAC as ligand and membranes prepared from activated T cells). Specific binding is preferably defined as the total binding (e.g., total cpm on filters) minus the non-specific binding. Non-specific binding is defined as the amount of cpm still detected in the presence of excess unlabeled competitor (e.g., IP-10, Mig, I-TAC). If desired, membranes prepared from cells which express recombinant CXCR3 can be used in the described assay.

The capacity of compounds to antagonize CXCR3 function can also be determined in a leukocyte chemotaxis assay using suitable cells. Suitable cells include, for example, cell lines, recombinant cells or isolated cells which express CXCR3 and undergo CXCR3 ligand-induced (e.g., IP-10, Mig, I-TAC) chemotaxis. In one example, CXCR3-expressing recombinant L1.2 cells (see, e.g., Campbell et al. J Cell Biol, 134:255-266 (1996)) or activated T cells, can be used in a modification of a transendothelial migration assay (Carr, M. W., et al., Proc. Natl Acad Sci, USA, (91):3652 (1994)). T cells can be isolated from whole blood by suitable methods, for example, density gradient centrifugation and positive or preferably negative selection with specific antibodies and activated using, for example, mitogens, anti-CD3 or cytokines (e.g., IL-2). The endothelial cells used in this assay are preferably the endothelial cell line, ECV 304, obtained from the European Collection of Animal Cell Cultures (Porton Down, Salisbury, U.K.). Endothelial cells can be cultured on 6.5 mm diameter Transwell culture inserts (Costar Corp., Cambridge, Mass.) with 3.0 μm pore size. Culture media for the ECV 304 cells can consist of M199+10% FCS, L-glutamine, and antibiotics. The assay media can consist of equal parts RPMI 1640 and M199 with 0.5% BSA. Two hours before the assay, 2×10⁵ ECV 304 cells can be plated onto each insert of the 24-well Transwell chemotaxis plate and incubated at 37° C. Chemotactic factors such as IP-10, Mig, I-TAC (commercially available from Peprotech, Rocky Hill, N.J., for example) diluted in assay medium can be added to the 24-well tissue culture plates in a final volume of 600 μL. Endothelial-coated Transwells can be inserted into each well and 10⁶ cells of the leukocyte type being studied are added to the top chamber in a final volume of 100 μL of assay medium. The plate can then be incubated at 37° C. in 5% CO₂/95% air for 1-2 hours. The cells that migrate to the bottom chamber during incubation can be counted, for example using flow cytometry. To count cells by flow cytometry, 500 μL of the cell suspension from the lower chamber can be placed in a tube and relative counts can be obtained for a set period of time, for example, 30 seconds. This counting method is highly reproducible and allows gating on the leukocytes and the exclusion of debris or other cell types from the analysis. Alternatively, cells can be counted with a microscope. Assays to evaluate chemotaxis inhibitors can be performed in the same way as control experiment described above, except that antagonist solutions, in assay media containing up to 1% of DMSO co-solvent, can be added to both the top and bottom chambers prior to addition of the cells. Antagonist potency can be determined by comparing the number of cells that migrate to the bottom chamber in wells which contain antagonist, to the number of cells which migrate to the bottom chamber in control wells. Control wells can contain equivalent amounts of DMSO, but no antagonist. If desired, the endothelial cells can be omitted from the described chemotaxis assay and ligand-induced migration across the Transwell insert can be measured.

The activity of an antagonist of CXCR3 function can also be assessed by monitoring cellular responses induced by active receptor, using suitable cells expressing receptor. For instance, exocytosis (e.g., degranulation of cells leading to release of one or more enzymes or other granule components, such as esterases (e.g., serine esterases), perforin, and/or granzymes), inflammatory mediator release (such as release of bioactive lipids such as leukotrienes (e.g., leukotriene C₄)), and respiratory burst, can be monitored by methods known in the art or other suitable methods (see e.g., Taub, D. D. et al., J. Immunol., 155: 3877-3888 (1995), regarding assays for release of granule-derived serine esterases; Loetscher et al., J. Immunol., 156: 322-327 (1996), regarding assays for enzyme and granzyme release; Rot, A. et al., J. Exp. Med., 176: 1489-1495 (1992) regarding respiratory burst; Bischoff, S. C. et al., Eur. J. Immunol., 23: 761-767 (1993) and Baggiolini, M. and C. A. Dahinden, Immunology Today, 15: 127-133 (1994)).

In one embodiment, an antagonist of CXCR3 is identified by monitoring the release of an enzyme upon degranulation or exocytosis by a cell capable of this function. Cells expressing CXCR3 can be maintained in a suitable medium under suitable conditions, and degranulation can be induced. The cells are contacted with an agent to be tested, and enzyme release can be assessed. The release of an enzyme into the medium can be detected or measured using a suitable assay, such as in an immunological assay, or biochemical assay for enzyme activity.

The medium can be assayed directly, by introducing components of the assay (e.g., substrate, co-factors, antibody) into the medium (e.g., before, simultaneous with or after the cells and agent are combined). The assay can also be performed on medium which has been separated from the cells or further processed (e.g., fractionated) prior to assay. For example, convenient assays are available for enzymes, such as serine esterases (see e.g., Taub, D. D. et al., J. Immunol., 155: 3877-3888 (1995) regarding release of granule-derived serine esterases).

In another embodiment, cells expressing CXCR3 are combined with a ligand of CXCR3 or promoter of CXCR3 function, an agent to be tested is added before, after or simultaneous therewith, and degranulation is assessed. Inhibition of ligand- or promoter-induced degranulation is indicative that the agent is an inhibitor of mammalian CXCR3 function.

In a preferred embodiment, the antagonist of CXCR3 function does not significantly inhibit the function of other chemokine receptors (e.g., CCR1, CXCR1, CCR3). Such CXCR3-specific antagonists can be identified by suitable methods, such as by suitable modification of the methods described herein. For example, cells which do not express CXCR3 (CXCR3⁻) but do express one or more other chemokine receptors (e.g., CCR1, CXCR1, CCR3) can be prepared or identified using suitable methods (e.g., transfection, antibody staining, western blot, RNAse protection). Such cells or cellular fractions (e.g., membranes) obtained from such cells can be used in a suitable binding assay. For example, when a cell which is CXCR3⁻ and CCR3⁺ is chosen, the CXCR3 antagonist can be assayed for the capacity to inhibit the binding of a suitable CCR3 ligand (e.g., RANTES) to the cell or cellular fraction, as described herein.

In another preferred embodiment, the antagonist of CXCR3 function is an agent which binds CXCR3. Such CXCR3-binding antagonists can be identified by suitable methods, for example, in binding assays employing a labeled (e.g., enzymatically-labeled (e.g., alkaline phosphatase, horse radish peroxidase), biotinylated, radio-labeled (e.g., ³H, ¹⁴C, ¹²⁵I)) antagonist.

In another preferred embodiment, the antagonist of CXCR3 function is an agent which can inhibit the binding of a (i.e., one or more) CXCR3 ligand to CXCR3, such as an agent which can inhibit the binding of human Mig, IP-10 and/or I-TAC to human CXCR3.

In a particularly preferred embodiment, the antagonist of CXCR3 function is an agent which can bind to CXCR3 and thereby inhibit the binding of a (i.e., one or more) CXCR3 ligand to CXCR3 (e.g., human CXCR3).

### Methods of Therapy

As used herein, the term “graft” refers to organs and/or tissues which can be obtained from a first mammal (a donor) and transplanted into a second mammal (a recipient), preferably a human. The term “graft” encompasses, for example, skin, eye or portions of the eye (e.g., cornea, retina, lens), muscle, bone marrow or cellular components of the bone marrow (e.g., stem cells, progenitor cells), heart, lung, heart-lung (e.g., heart and a single lung, heart and both lungs), liver, kidney, pancreas (e.g., islet cells, β-cells), parathyroid, bowel (e.g., colon, small intestine, duodenum), neuronal tissue, bone and vasculature (e.g., artery, vein). A graft can be obtained from a suitable mammal (e.g., human, pig, baboon, chimpanzee), or under certain circumstances a graft can be produced in vitro by culturing cells, for example, embryonal cells, fetal cells, skin cells, blood cells and bone marrow cells which were obtained from a suitable mammal. A graft is preferably obtained from a human. In one embodiment, the graft is other than a skin graft.

The graft can be obtained from a genetically modified animal or can be modified (e.g., genetically, chemically, physically) using any suitable method. In one embodiment, a modified graft having reduced capacity to express a ligand for CXCR3 (e.g., IP-10, Mig and/or I-TAC), relative to a suitable control (e.g., an unmodified or wild type graft) is transplanted. Such a graft can, for example, carry a targeted mutation in a gene encoding a CXCR3 ligand. Targeted mutations can be produced using a variety of suitable methods. For example, a targeted mutation can be introduced into the genome of embryonic stem cells or zygotes using standard techniques. The resulting mutant cells can develop into animals carrying the targeted mutation (e.g., heterozygous or homozygous). For example, pigs or other animals which express human MHC antigens and which are homozygous for a targeted mutation in a gene encoding a CXCR3 ligand (e.g., IP-10) can be created. The organs from such animals (xenografts) can be transplanted into a human.

An “allograft”, as the term is used herein, refers to a graft comprising antigens which are allelic variants of the corresponding antigens found in the recipient. For example, a human graft comprising an MHC class II antigen encoded by the HLA-DRB1*0401 allele is an allograft if transplanted into a human recipient whose genome does not comprise the HLA-DRB1*0401 allele.

In one embodiment, the method of inhibiting (reducing or preventing) graft rejection comprises administering an effective amount of an (i.e., one or more) antagonist of CXCR3 function to a recipient of a graft. In another embodiment, the method of inhibiting graft rejection comprises administering an effective amount of an antagonist of CXCR3 function to a recipient of an allograft. In a preferred embodiment, the method comprises administering an effective amount of an antagonist of CXCR3 function to a recipient of a cardiac allograft.

In another embodiment, the antagonist of CXCR3 function is selected from the group consisting of small organic molecules, natural products, peptides, peptidomimetics and proteins, wherein said proteins are not chemokines or mutants or analogues thereof.

In a preferred embodiment, the invention provides a method for inhibiting (reducing or preventing) graft rejection comprising administering to a graft recipient an effective amount of an antagonist of CXCR3 function and an effective amount of an (i.e., one or more) additional therapeutic agent, preferably an immunosuppressive agent. Advantageously, the rejection-inhibiting effects of CXCR3 antagonists and immunosuppressive agents can be additive or synergistic, and can result in permanent engraftment.

A further benefit of co-administration of a CXCR3 antagonist and an immunosuppressive agent is that the dose of immunosuppressive agent required to inhibit graft rejection can be reduced to sub-therapeutic levels (e.g., a dose that does not inhibit graft rejection when administered as the sole therapeutic agent). The ability to reduce the dose of the immunosuppressive agent can greatly benefit the graft recipient as many immunosuppressive agents have severe and well-known side effects including, for example, increased incidence of infection, increased incidence of certain malignancies, diabetes mellitus, neurotoxicity, nephrotoxicity, hyperlipidemia, hypertension, hirsutism, gingival hyperplasia, impaired wound healing, lymphopenia, jaundice, anemia, alopecia and thrombocytopenia (Spencer, C. M., et al., Drugs, 54(6):925-975 (1997); Physicians Desk Reference, 53^{rd} Edition, Medical Economics Co., pp. 2081-2082 (1999)).

The term “immunosuppressive agent”, as used herein, refers to compounds which can inhibit an immune response. The immunosuppressive agent used in the invention can be a novel compound or can be selected from the compounds which are known in the art, for example, calcineurin inhibitors (e.g., cyclosporin A, FK-506), IL-2 signal transduction inhibitors (e.g., rapamycin), glucocorticoids (e.g., prednisone, dexamethasone, methylprednisolone, prednisolone), nucleic acid synthesis inhibitors (e.g., azathioprine, mercaptopurine, mycophenolic acid) and antibodies to lymphocytes or antigen-binding fragments thereof (e.g., OKT3, anti-IL2 receptor). Novel immunosuppressive agents can be identified by those of skill in the art using suitable methods, for example, screening compounds for the capacity to inhibit antigen-dependent T cell activation.

The immunosuppressive agent used for co-therapy (e.g., co-administration with an antagonist of CXCR3 function) is preferably a calcineurin inhibitor. More preferably the immunosuppressive agent used for co-therapy is cyclosporin A.

When the graft is bone marrow, cells (e.g., leukocytes) derived from the graft can mount an immune response directed at the recipient's organs and tissues. Such a condition is referred to in the art as graft versus host disease (GVHD). Administration of an antagonist of CXCR3 function with or without an additional therapeutic agent (e.g., immunosuppressive agent, hematopoietic growth factor) can inhibit GVHD. Accordingly, in another embodiment, the invention provides a method of inhibiting (reducing or preventing) GVHD in a bone marrow graft recipient comprising administering an effective amount of an antagonist of CXCR3 function. In an additional embodiment, the method of inhibiting GVHD comprises the administration of an effective amount of an antagonist of CXCR3 function and an effective amount of one or more additional therapeutic agents, for example, an immunosuppressive agent.

In another embodiment, the method of inhibiting GVHD comprises the administration of an effective amount of an antagonist of CXCR3 function, which is selected from the group consisting of small organic molecules, natural products, peptides, peptidomimetics and proteins, wherein said proteins are not chemokines or mutants or analogues thereof.

The invention further relates to the use of an antagonist of CXCR3 function for the manufacture of a medicament for inhibiting graft rejection (e.g., acute rejection, chronic rejection) as described herein. The invention also relates to a medicament for inhibiting graft rejection (e.g., acute rejection, chronic rejection) wherein said medicament comprises an antagonist of CXCR3 function.

A “subject” is preferably a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

An effective amount of the antagonist of CXCR3 function can be administered to a subject to inhibit (reduce or prevent) graft rejection. For example, an effective amount of the antagonist of CXCR3 function can be administered before, during and/or after transplant surgery or other medical procedure for introduction of a graft to a recipient (e.g., transfusion).

When co-administration of an antagonist of CXCR3 function and an additional therapeutic agent is indicated or desired for inhibiting graft rejection, the antagonist of CXCR3 function can be administered before, concurrently with or after administration of the additional therapeutic agent. When the antagonist of CXCR3 function and additional therapeutic agent are administered at different times, they are preferably administered within a suitable time period to provide substantial overlap of the pharmacological activity (e.g., inhibition of CXCR3 function, immunosuppression) of the agents. The skilled artisan will be able to determine the appropriate timing for co-administration of an antagonist of CXCR3 function and an additional therapeutic agent depending on the particular agents selected and other factors.

An “effective amount” of a CXCR3 antagonist is an amount sufficient to achieve a desired therapeutic and/or prophylactic effect, such as an amount sufficient to inhibit graft rejection. For example, an effective amount is an amount sufficient to inhibit a (i.e., one or more) function of CXCR3 (e.g., CXCR3 ligand-induced leukocyte migration, CXCR3 ligand-induced integrin activation, CXCR3 ligand-induced transient increase in the concentration of intracellular free calcium [Ca²⁺]₁ and/or CXCR3 ligand-induced secretion (e.g., degranulation) of proinflammatory mediators), and thereby inhibit graft rejection. An “effective amount” of an additional therapeutic agent (e.g., immunosuppressive agent) is an amount sufficient to achieve a desired therapeutic and/or prophylactic effect (e.g., immunosuppression).

The amount of agent (e.g., CXCR3 antagonist, additional therapeutic agent) administered to the individual will depend on the characteristics of the individual, such as general health, age, sex, body weight and tolerance to drugs as well as the degree, severity and type of rejection. The skilled artisan will be able to determine appropriate dosages depending on these and other factors. Typically, an effective amount can range from about 0.1 mg per day to about 100 mg per day for an adult. Preferably, the dosage ranges from about 1 mg per day to about 100 mg per day. Antibodies and antigen-binding fragments thereof, particularly human, humanized and chimeric antibodies and antigen-binding fragments can often be administered less frequently than other types of therapeutics. For example, an effective amount of such an antibody can range from about 0.01 mg/kg to about 5 or 10 mg/kg administered daily, weekly, biweekly, monthly or less frequently.

The agent (e.g., CXCR3 antagonist, additional therapeutic agent) can be administered by any suitable route, including, for example, orally (e.g., in capsules, suspensions or tablets) or by parenteral administration. Parenteral administration can include, for example, intramuscular, intravenous, intraarticular, intraarterial, intrathecal, subcutaneous, or intraperitoneal administration. The agent (e.g., CXCR3 antagonist, additional therapeutic agent) can also be administered orally (e.g., dietary), transdermally, topically, by inhalation (e.g., intrabronchial, intranasal, oral inhalation or intranasal drops) or rectally. Administration can be local or systemic as indicated. The preferred mode of administration can vary depending upon the particular agent (e.g., CXCR3 antagonist, additional therapeutic agent) chosen, however, oral or parenteral administration is generally preferred.

The agent (e.g., CXCR3 antagonist, additional therapeutic agent) can be administered as a neutral compound or as a salt. Salts of compounds containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium and the like.

The antagonist of CXCR3 function can be administered to the individual as part of a pharmaceutical composition for inhibition of graft rejection comprising a CXCR3 antagonist and a pharmaceutically or physiologically acceptable carrier. Pharmaceutical compositions for co-therapy can comprise an antagonist of CXCR3 function and one or more additional therapeutic agents. An antagonist of CXCR3 function and an additional therapeutic agent can be components of separate pharmaceutical compositions which can be mixed together prior to administration or administered separately. Formulation will vary according to the route of administration selected (e.g., solution, emulsion, capsule). Suitable pharmaceutical or physiological carriers can contain inert ingredients which do not interact with the antagonist of CXCR3 function and/or additional therapeutic agent. Standard pharmaceutical formulation techniques can be employed, such as those described in Remington's Pharmaceutical Sciences, Mack Publishing Company, Easton, Pa. Suitable carriers for parenteral administration include, for example, sterile water, physiological saline, bacteriostatic saline (saline containing about 0.9% benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

### Diagnostic Methods

The invention also relates to a method for diagnosing graft rejection. According to the method, expression of chemokine receptors (e.g., CXCR3, CCR1) and/or chemokines (e.g., CXCR3 ligands (e.g., IP-10, Mig, I-TAC), CCR1 ligands (e.g., MIP-1α, RANTES, MCP-2, MCP-3, MCP-4, CKbeta8, CKbeta8-1, leukotactin-1, HCC-1, MPIF-1)) by and/or in grafts can be assessed to diagnose graft rejection. As described herein, a study was conducted to assess the expression of chemokine receptors and chemokines (e.g., CXCR3, CCR1 and their respective ligands) in human cardiac allografts (Example 3). In the study, expression of chemokines or chemokine receptors was assessed by performing reverse transcription-polymerase chain reaction (RT-PCR) or immunohistochemistry on biopsies of human cardiac allografts. The results of the study revealed that expression of RANTES or IP-10 by the graft is associated with rejection, and that rejection is associated with the presence of CXCR3⁺ or CCR1⁺ infiltrates. There was no evidence of rejection in cardiac allografts which did not contain CXCR3⁺ or CCR1⁺ infiltrates.

In one embodiment, the method comprises assessing (detecting or measuring) a graft-expressed chemokine (e.g., a ligand for CCR1 (e.g., RANTES), a ligand for CXCR3 (e.g., IP-10)) in, for example, a graft or sample thereof. Any suitable method can be used to assess expression. For example, immunohistochemistry or RT-PCR can be performed on a sample of the graft obtained by biopsy. Suitable in vivo methods of detection can also be used. In one example, a radio-labeled antibody which binds a chemokine (e.g., RANTES, IP-10) can be administered to a graft recipient and antibody-chemokine complex formation can be detected (e.g., radiologically). The presence of graft-expressed chemokine (e.g., RANTES, IP-10) or of an elevated amount of graft-expressed chemokine relative to a suitable control is indicative of rejection. Suitable controls include, for example, non-rejecting grafts and normal tissue.

In another embodiment, the method comprises assessing (detecting or measuring) infiltration of a graft by CXCR3⁺ and/or CCR1⁺ cells (e.g., CD3⁺ T cells). Any suitable method (in vivo method, in vitro method) can be used to assess infiltration by CXCR3⁺ and/or CCR1⁺ cells. For example, an aspirated sample of the graft can be analyzed by flow cytometry using antibodies which bind CXCR3 or CCR1 or a graft sample obtained by biopsy (e.g., cardiac biopsy) can be analyzed by immunohistochemistry. The presence of infiltrating CXCR3⁺ and/or CCR1⁺ cells (e.g., CD3⁺ T cells) relative to a suitable control (e.g., an autologous organ) is indicative of rejection.

The diagnostic methods can be used in diagnoses to assess rejection of any graft, and can be used to guide therapy or to monitor the efficacy of therapy (e.g., immunosuppressive therapy, initiate anti-viral therapy). For example, graft recipients are routinely treated with immunosuppressive drugs to inhibit graft rejection. However, as discussed herein, such therapy can have severe side effects and the dose of immunosuppressive agents must be carefully monitored. In one application of the method, the presence of a CXCR3⁺ infiltrate (e.g., CD3⁺ T cells) in a graft biopsy is assessed. The presence of such an infiltrate can be indicative of acute rejection. Accordingly, immunosuppressive therapy can be increased. In another example, a CXCR3⁺ infiltrate is not detected in a cardiac allograft biopsy of a graft that is not functioning well as assessed by, for example, electrocardiogram (EKG). In this situation, graft dysfunction is not the result of acute rejection but can be the result of, for example, viral infection (e.g., cytomegalovirus). Thus, immunosuppressive therapy can be decreased or discontinued to allow the recipient's immune system to fight the infection and thereby improve graft function and/or appropriate therapy (e.g., anti-viral therapy) can be administered. Preferably, the diagnostic method is used to assess rejection of a solid organ graft (e.g., cardiac graft).

The present invention will now be illustrated by the following Examples, which are not intended to be limiting in any way.

### EXAMPLES

### Example 1

### CXCR3 Targeting and Cardiac Transplantation

Methods

Mice. CXCR3 KO mice (also referred to as CXCR3−/−) which are homozygous for a targeted deletion of the coding region of the CXCR3 gene were provided by Craig Gerard (Children's Hospital, Boston, Mass.) and bred at Millennium Pharmaceuticals, Inc. (Cambridge, Mass.). The targeted deletion was produced in BALB/c mice (H-2^{d}) and bred into C56BL/6 mice (H-2^{b}). Both BALB/c CXCR3 KO and C57BL/6 CXCR3 KO mice were used in the study. IP-10 KO mice (also referred to as IP-10−/−, strain B6/129, H-2^{b}) which are homozygous for a targeted gene disruption of the gene encoding IP-10 were provided by Andrew Luster (Massachusetts General Hospital, Boston, Mass.). All other mice were obtained from Jackson Laboratory (Bar Harbor, Me.). These included donor strains and control recipients (BALB/c, C57BL/6, B6/129). BALB/c differs from C57BL/6 and B6/129 at both class I and class II major histocompatibility complex (MHC) loci.

Mouse cardiac allografting (Mottram, P. L. et al., Transplantation 59:559-565 (1995); Hancock, W. W., et al., Proc. Natl. Acad. Sci (USA), 93:13967-13972 (1996)) was performed with the aid of an operating microscope (Nikon, 4× to 38× magnification) under clean conditions.

Preparation of the donor heart. Donor mice were anesthetized with Nembutal (50 mg/10 g body weight) and Atropine sulfate (0.17 mg/100 g body weight) i.p.; additional anaesthesia with Methoxyflurane supplementation was administered via a face mask as required during the procedure. Mice were shaved and cleansed with 70% alcohol. A midline abdominal incision was made in the donor animal and 1 ml of a 10% solution of heparin in saline was injected into the inferior vena cava. The incision was then extended cephalic to open the chest through a median sternotomy. The thorax was opened. The inferior vena cava was ligated with 6-0 silk and divided inferior to the tie. The superior vena cava was then similarly ligated and divided superior to the tie. The aorta and pulmonary artery were separated and divided as far distally as possible. At this point, blood was evacuated from the heart by applying pressure with applicator sticks. The aorta was transected just proximal to the brachiocephalic artery and the main pulmonary artery transected just proximal to its bifurcation. The pulmonary veins were then ligated and divided en mass and the heart placed in iced saline.

Preparation of the recipient. After being anesthetized in the same way as the donor, the recipient was brought under the microscope, a midline abdominal incision was made, and segments of the aorta and vena cava below the renal vessels were dissected free, but not separated from each other, over a length of about 2 mm. A clamp was placed on the proximal aorta and vena cava, and a distal tie of 6-0 silk was placed around both the aorta and vena cava in preparation for later occlusion of the vessels.

Transplantation of the heart. The tie that had been placed around the distal aorta and vena cava was secured by means of a single knot. An aortotomy and a venotomy in the vena cava were made adjacent to one another. The donor heart was then removed from the chilled saline, and the donor aorta and pulmonary artery were joined end-to-side to the recipient aorta and vena cava, respectively, with running suture, using 10-0 tipped with a BV-3 needle. Since the anastomoses were done adjacent to one another, the side of the pulmonary artery-cava suture line next to the aortic anastomosis was sutured from the inside with an everting running suture. During this period, chilled saline was dripped on the ischemic heart at frequent intervals. After the completion of the anastomoses, the inferior vascular occluding tie was released first, thus filling the inferior vena cava and donor pulmonary artery with recipient venous blood. Upon release of the proximal occluding tie, the aorta and coronary arteries of the transplant were perfused with oxygenated recipient blood. Blood loss was minimized by gradual release of the proximal tie. Warm saline was used externally to warm the heart immediately after establishing coronary perfusion. With warming and coronary perfusion, the heart began to fibrillate and usually within a few minutes it reverted spontaneously to a sinus rhythm. Occasionally, cardiac massage was required to re-establish a normal beat. The intestines were placed carefully back into the abdominal cavity around the auxiliary heart, and the abdomen was closed with a single running suture to all layers (saline with antibiotic was used to wash the peritoneal cavity as needed). The mouse was then placed in a constant temperature at 35° C. for recovery from anesthesia.

Therapeutic intervention. The effect of cyclosporin A (CsA) (Sigma, St. Louis, Mo.) therapy (10 mg/kg/day, intraperitoneal injection) was tested by injecting recipient mice with CsA daily until rejection or for a maximum of 14 days, beginning on the day of transplantation. Two anti-IP-10 antibodies were used in the study. A monoclonal rat anti-mouse CRG (IP-10) (IgG1) was purchased from PharMingen (San Diego, Calif.) and a monoclonal hamster anti-mouse IP-10 (IgG) was provided by A. Luster (Massachusetts General Hospital, Boston, Mass.). Polyclonal goat anti-mouse Mig was purchased from PharMingen (San Diego, Calif.). Monoclonal antibody 4C4 (IgM) was produced by immunizing rats with transfected cells expressing mouse CXCR3 followed by fusion of spleenocytes to myeloma cells to produce hybridomas. mAb 4C4 binds mouse CXCR3 and inhibits chemotaxis of cells expressing recombinant CXCR3 induced by IP-10 or Mig, but does not inhibit chemotaxis induced by chemokines which do not bind CXCR3. Nonspecific rat IgG was used as an irrelevant control. All antibodies were administered (200 μg by intraperitoneal injection) to recipient mice at transplantation and every 48 hours thereafter for fourteen days.

Monitoring of allograft survival. Cardiac allograft survival was monitored twice daily by palpation of ventricular contractions through the abdominal wall (Mottram, P. L. et al., Transplantation, 59:559-565 (1995)), rejection was defined as the day of cessation of palpable heartbeat, and was verified by autopsy (Gerard, C, et al., J. Clin Invest., 100:2022-2027 (1997); Mottram, P. L. et al., Transplantation, 59.559-565 (1995)). Once cardiac graft function ceased, mice were anesthetized as above, and grafts were surgically excised, subdivided into portions for (a) formalin fixation, paraffin embedding and subsequent light microscopy examination, or (b) snap-frozen in liquid nitrogen and stored at −70° C. until processed for immunohistology or RNAse protection assays.

Immunopathology. For histology, paraffin sections were stained with hematoxylin and eosin (H&E) to evaluate graft morphology, and with Weigert's elastin stain so as to examine the extent of intimal proliferation in penetrating branches of myocardial arteries (a key feature of transplant arteriosclerosis) (Gerard, C, et al., J. Clin Invest. 100:2022-2027 (1997); Mottram, P. L., et al., Transplantation 59:559-565 (1995)). Chemokine and chemokine receptor mRNA expression was determined using RNAse protection assay kits (Pharmingen, San Diego, Calif.).

### Results

Allograft survival data (mean±SD) are summarized in Tables 1 and 2 (using 6-10 animals/group).

TABLE 1
Effect of CXCR3 KO or CXCR3-ligand KO on mouse cardiac
allograft survival

| | Strains | | | Survival | |
|---|---|---|---|---|---|
| | (Donor → | MHC | | (Mean ± | |
| # | Recipient) | mismatch | Therapy | SD, days) | probability |
| 1 | C57BL/6 → | class I & II | — | 7.7 ± 0.8 | |
| | BALB/c | | | | |
| 2 | C57BL/6 → | class I & II | — | 49.8 ± 1.2 | p < 0.0001 |
| | CXCR3 KO | | | | (cf. #1) |
| | (knockout on | | | | |
| | BALB/c) | | | | |
| 3 | C57BL/6 → | class I & II | rat IgG | 7.8 ± 0.9 | |
| | BALB/c | | | | |
| 4 | C57BL/6 → | class I & II | anti-IP-10 | 13.7 ± 0.5 | p < 0.01 |
| | BALB/c | | antibody | | (cf. #3) |
| 5 | C57BL/6 → | class I & II | anti-Mig | 12.9 ± 0.3 | p < 0.01 |
| | BALB/c | | antibody | | (cf. #3) |
| 6 | BALB/c → | class I & II | — | 7.4 ± 0.4 | |
| | C57BL/6 | | | | |
| 7 | BALB/c → | class I & II | — | 57.1 ± 1.4 | p < 0.001 |
| | CXCR3 KO | | | | (cf. #6) |
| | (knockout on | | | | |
| | C57BL/6) | | | | |
| 8 | BALB/c → | class I & II | — | 7.6 ± 0.5 | |
| | IP-10 KO | | | | |
| 9 | B6/129 → | class I & II | — | 7.4 ± 0.4 | |
| | BALB/c | | | | |
| 10 | IP-10 KO → | class I & II | — | 42.3 ± 0.9 | p < 0.001 |
| | BALB/c | | | | (cf. #9) |

p values were determined by the Mann-Whitney U test.

The results presented in Table 1 demonstrate that CXCR3⁺ cells contribute to the pathogenesis of allograft rejection. Disruption of CXCR3 function in a complete MHC mismatch significantly prolongs allograft survival (group 1 vs. 2). In addition, the administration of neutralizing antibodies which bind the CXCR3 ligands (anti-IP10 (group 4) and anti-Mig (group 5)) also extended the period of allograft survival. These results emphasize the importance of the CXCR3 pathway in graft rejection and demonstrate that disruption of receptor function was more effective at preventing rejection than inhibition of individual ligands (compare groups 1-5).

In further studies, C56BL/6 CXCR3 KO mice were use. These mice had been backcrossed to a homogenous genetic background (F5). The studies confirmed that inhibition of CXCR3 function can powerfully inhibit graft rejection (compare groups 6 and 7). In addition, inhibiting expression of IP-10 by the graft greatly extended the survival of cardiac grafts with complete MHC mismatch (compare groups 9 and 10).

In further studies the effect of treatment with anti-CXCR3 mAb 4C4 on cardiac allograft survival (BALB/c→C57BL/6) was evaluated. Allografts remained viable for about 1 week in control recipients. In contrast, grafts survived for at least three weeks in recipients treated with anti-CXCR3 mAb 4C4.

### Pathologic Findings

Cardiac allografts in the BALB/c→C57BL/6 combination using wild type recipients or CXCR3 KO recipients were removed after seven days, sectioned, stained (H&E) and studied. Severe rejection was evident in grafts removed from wild type recipients with acute vascular and cellular rejection. In contrast, grafts removed from CXCR3 KO recipients exhibited normal myocardium and vessels in conjunction with only focal small mononuclear cell collections. In the absence of therapeutic intervention, cellular rejection developed in grafts transplanted into CXCR3 KO recipients by about day 55.

TABLE 2
Effect of CXCR3 KO and low dose immunosuppression on mouse
cardiac allograft survival

| | Strains | | | Survival | |
|---|---|---|---|---|---|
| | (Donor → | MHC | | (Mean ± | |
| # | Recipient) | mismatch | Therapy | SD, days) | probability |
| 11 | BALB/c → | class I & II | — | 7.4 ± 0.5 | |
| | C57BL/6 | | | | |
| 12 | BALB/c → | class I & II | low CsA* | 10.4 ± 1.2 | |
| | C57BL/6 | | | | |
| 13 | BALB/c → | class I & II | — | 55.2 ± 2.2 | p < 0.001 |
| | CXCR3 KO | | | | vs. #1 |
| | (knockout on | | | | |
| | C57BL/6) | | | | |
| 14 | BALB/c → | class I & II | low CsA, | >100 | p < 0.001 |
| | CXCR3 KO | | 14 days | | vs. #2 |
| | (knockout on | | | | |
| | C57BL/6) | | | | |

p values were determined by the Mann-Whitney U test.
*CsA administered until death or for a maximum of 14 days.

The results presented in Table 2 demonstrate that a brief course of cyclosporin A (10 mg/kg/d) following transplantation induced only a minor prolongation of allograft survival in control mice (about 3 days) as compared with untreated recipients (compare groups 11 and 12). However, the same dose of CsA in CXCR3 KO mice (for a maximum of 14 days) led to permanent engraftment in all recipients (group 14). The beneficial actions of some experimental agents can be undermined by concomitant immunosuppression. However, CsA and inhibition of CXCR3 function are additive or synergistic in efficacy.

### Example 2

### CXCR3 and Chronic Rejection in Cardiac Allograft Recipients

Administration of CD4 monoclonal antibody (mAb) can prolong the survival of cardiac allografts in the described murine model (Mottram et al., Transplantation 59:559-565 (1995)). However, the extended survival of grafts in anti-CD4 treated animals is complicated by the development of chronic rejection with florid transplant arteriosclerosis (Hancock et al., Nature Medicine 4:1392-1396 (1998)).

### Methods

Cardiac allografts derived from BALB/c donors were transplanted into CXCR3 KO or CXCR3+/+ control mice (C57BL/6) as described in Example 1.

Immunosuppression. CD4 mAb (GK1.5, American Type Culture Collection, Manassas, Va.; Accession No. TIB-207) was administered four times to CXCR3+/+ allograft recipients (6/group); 250 μg by intraperitoneal injection on day 0 (time of transplantation) and on subsequent days 1, 2 and 3. Cyclosporin A was administered to CXCR3 KO graft recipients as described in Example 1 (group 14).

Monitoring of chronic rejection. Cardiac allograft survival was monitored twice daily by palpation of ventricular contractions through the abdominal wall. All grafts survived to day 60, and the readout was morphologic examination, particularly, the extent of development of transplant arteriosclerosis. Accordingly, grafts were fixed in formalin, embedded in paraffin and sections counterstained with Weigert's elastin stain. Cardiac grafts were removed from CXCR3 KO recipients (group 14) at 100 days post transplant for analysis. All intramyocardial arteries were scored for the extent of intimal proliferation as <5% occlusion (0); 5-20% occlusion (1); 21-40% occlusion (2); 41-60% occlusion (3); 61-80% occlusion (4); or 81-100% occlusion (5) (Murphy et al., Transplantation 64:14-19 (1997)).

### Results

Results of scoring of vessels within cardiac allografts (6 grafts/group) and statistical evaluation (Mann-Whitney U test) are presented in Table 3.

TABLE 3
Effect of CXCR3 KO on development of transplant-associated
arteriosclerosis

| | Strains | | | | |
|---|---|---|---|---|---|
| | (Donor → | | Total | Vessel Score | |
| # | Recipient) | Therapy | Vessels | (mean ± SD) | p value |
| 15 | BALB/c → | anti-CD4 | 32 | 2.7 ± 0.7 | |
| | C57BL/6 | antibody | | | |
| 14 | BALB/c → | low CsA, | 37 | 0.2 ± 0.2 | p < 0.001 |
| | CXCR3 KO | 14 days | | | vs. #1 |
| | (knockout on | | | | |
| | C57BL/6) | | | | |

p values were determined by the Mann-Whitney U test.

Grafts harvested at day 100 from CXCR3 KO recipients showed only a minor mononuclear cell infiltrate and no evidence of transplant-associated arteriosclerosis. These findings are in contrast to the severe arteriosclerosis observed in grafts removed from wild type allograft recipients which were treated with high dose CsA (30 mg/kg/d) or CD4 mAb therapy (Mottram, P. L., Han, W. R., et al., “Increased expression of IL-4 and IL-10 and decreased expression of IL-2 and IFN-γ in long-surviving mouse heart allografts after brief CD4-monoclonal antibody therapy,” Transplantation 59:559-565 (1995); Hancock, W. W., Buelow, R., et al., “Antibody-induced transplant arteriosclerosis is prevented by graft expression of anti-oxidant and anti-apoptotic genes,” Nature Medicine 4: 1392-1396 (1998)).

The results demonstrate that inhibition of CXCR3 function blocks the development of transplant-associated atherosclerosis and the development of other features of chronic rejection.

### Example 3

### Expression of Chemokines and Chemokine Receptors in Human Cardiac Allografts: Association with CD3+ T Cell Infiltrates and Rejection

Chemokines function in the recruitment as well as in the activation of leukocytes. However, little is reported on the expression or function of chemokines in human allografts. In this study the expression of RANTES (Ran), MCP-1, Mig, IP-10, SDF-1, lymphotactin (Lt), and eotaxin mRNA in human cardiac allograft biopsies was examined using semiquantitative RT-PCR. The expression of the chemokine receptors CCR1, CCR3, CCR5 and CXCR3 was examined in separate biopsies by immunohistochemistry. Biopsies taken at the same time as the study biopsies were examined for the clinicopathologic diagnosis of rejection using the ISHLT scoring system. A total of 44 biopsies (n=44 patients) were examined by RT-PCR. Rejection grade 1A was found in 6 biopsies and grade 2 in 5 biopsies. No rejection was found in 33 biopsies. The expression of chemokines are summarized in Table 4 as the percentage of biopsies expressing the chemokine.

TABLE 4

| | SDF-1 | Eotaxin | Lt | IP-10 | Ran | Mig | MCP-1 |
|---|---|---|---|---|---|---|---|
| no | 61% | 45% | 54% | 29% | 30% | 6% | 6% |
| rejection | | | | | | | |
| rejection | 64% | 55% | 36% | 64% | 55% | 9% | 0% |

A total of 27 biopsies were examined by immunohistochemistry. CCR3 was found on resident cells and on infiltrating mononuclear cells in most biopsies, without any association with rejection. CCR5 was found in 41% of biopsies on occasionally infiltrating leukocytes, and was not associated with the histological diagnoses. In contrast, the expression of CXCR3 and CCR1 was associated with the presence of CD3⁺ T cell infiltrates (p<0.05). Furthermore, in the absence of CXCR3- or CCR1-expressing infiltrates, there was no evidence of rejection. In addition, in most biopsies with CD3⁺ T cell infiltrates and evidence of rejection, CXCR3-expressing and CCR1-expressing cells were present. Thus, the expression of eotaxin, SDF-1 and lymphotactin, as well as of CCR3, is common in human cardiac allograft biopsies and is not associated with the clinicopathologic diagnosis. In contrast, RANTES and IP-10, as well as their respective receptors, CCR1 and CXCR3, can be associated with acute rejection.

### Example 4

### Immune Response of CXCR3 KO Mice Methods

In vitro T-cell proliferation responses. Mixed lymphocyte responses (MLRs) were assessed by culturing responder splenocytes (isolated from CXCR3 KO (CXCR3−/− on C57BL/6 background) or wild type (WT, CXCR3+/+ on C57BL6 background) mice) with mitomycin-C treated allogeneic stimulator splenocytes (isolated from BALB/c mice) in RPMI-1640 medium containing 5% FBS, 1% penicillin/streptomycin and 5×10⁻⁵ M 2-mercaptoethanol, in 96 flat-bottom wells (Gao, W. et al., J. Clin. Invest. 105:35-44 (2000); Mottram, P. L. et al., Transplantation 59:559-565 (1995), the entire teachings of both of which are incorporated herein by reference). Cultures were incubated at 37° C. in 5% CO₂ for 3 to 5 days and were pulsed with [³H]thymidine for 6 hours before harvesting. The mean amount of radioactivity incorporated into cells (counts per minute) and standard deviation were calculated using 12 wells per group.

Some cultures contained anti-CXCR3 mAb 4C4 or an IgM control antibody. When present, the antibodies were used at a concentration of 10, 1 or 0.1 μg/ml.

Mitogen-induced proliferation of T cells was measured using Concanavalin-A (Con-A, a T cell mitogen) (Mottram, P. L. et al., Transplantation 59:559-565 (1995)). Splenocytes isolated from CXCR3 KO (CXCR3−/− on C57BL/6 background) or wild type (WT, CXCR3+/+ on C57BL6 background) mice were cultured in 96 well flat-bottom plates in RPMI-1640 medium containing 5% FBS, 1% penicillin/streptomycin and 5×10⁻⁵ M 2-mercaptoethanol and 1.25, 2.5, 5 or 10 μg/ml concanavalin-A (Con-A, Sigma Chemical Co., St. Louis, Mo.). The cultures were incubated at 37° C. in 5% CO₂ for 72 hours and were pulsed with [³H]thymidine for 6 hours before harvesting. The mean amount of radioactivity incorporated into cells (counts per minute) and standard deviation were calculated using 12 wells per group.

Statistical analysis. Data from the proliferation assays were compared using the Mann-Whitney U test.

### Results

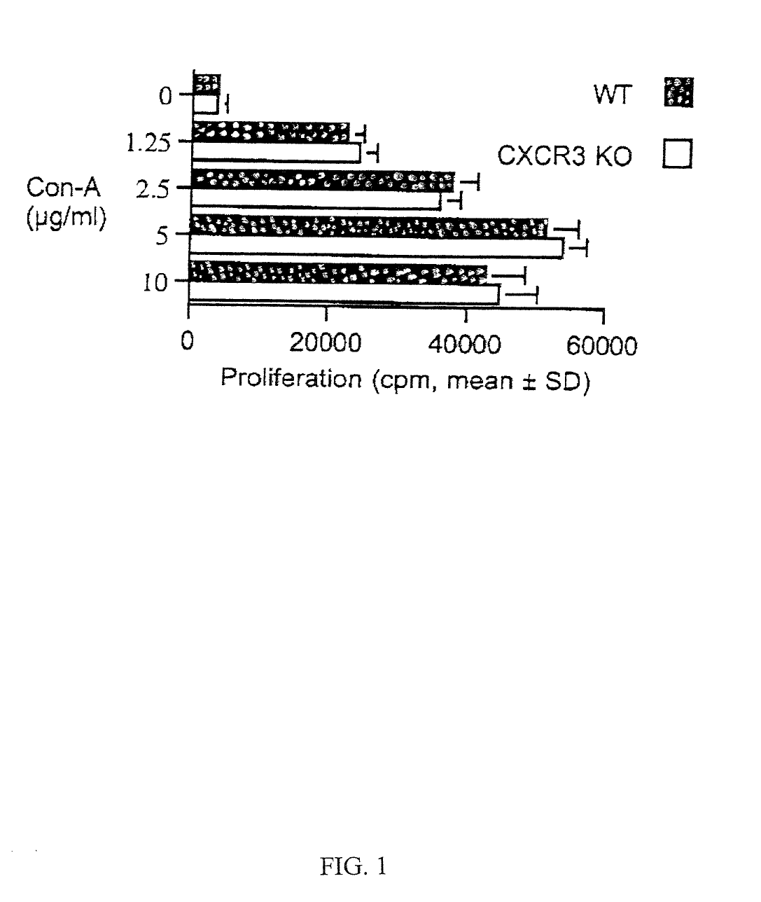

The results of the in vitro T cell proliferation studies are presented in FIGS. 1-3. T cell proliferation in mitogen-stimulated (Con-A-stimulated) cultures of splenocytes that were isolated from CXCR3−/− (C57BL/6) mice was about equivalent to T cell proliferation in Con-A-stimulated cultures of splenocytes from wild type (WT, CXCR3+/+ on C57BL/6 background) mice (FIG. 1). The results indicate that CXCR3−/− (C57BL/6) mice have normal T cell proliferative responses upon stimulation with mitogen.

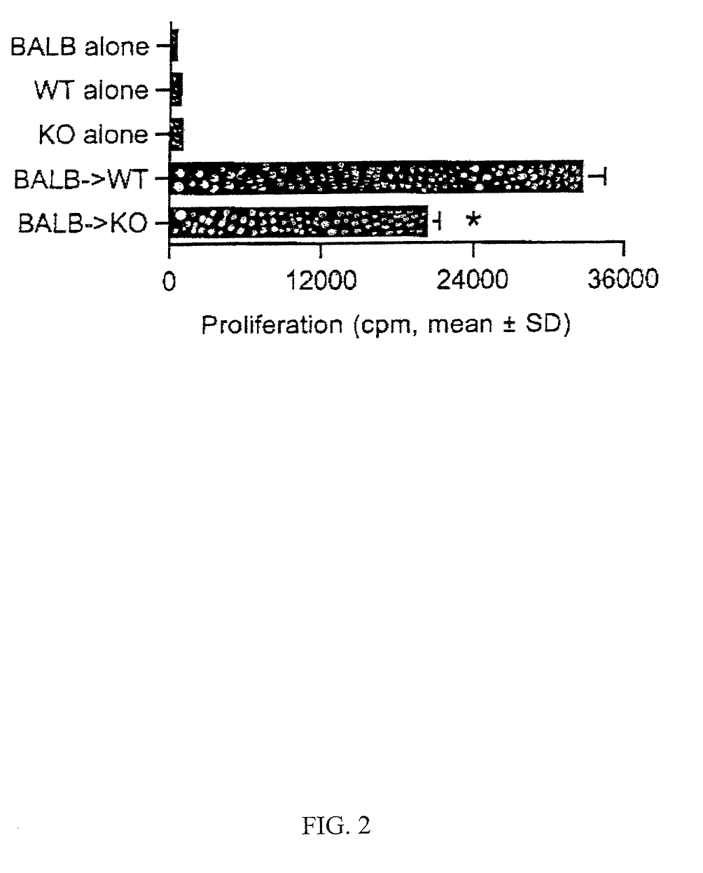

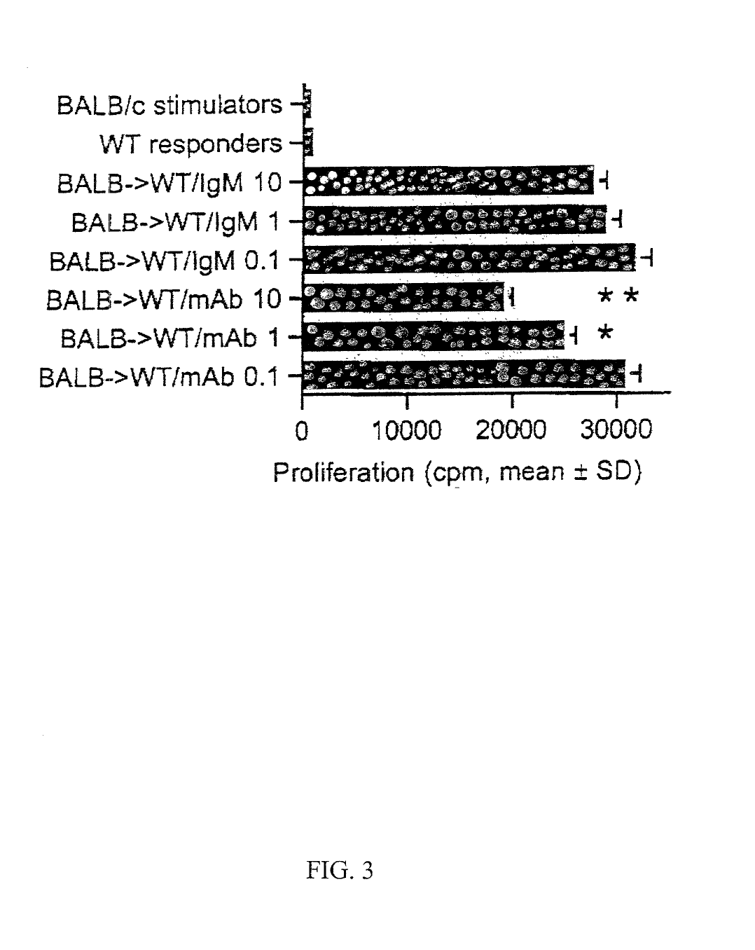

Cells isolated from CXCR3−/− (C57BL/6) mice also developed a robust response to allogeneic stimulator cells in MLR assays (FIG. 2). However, the overall magnitude of the response was significantly lower than that of wild type (WT, CXCR3+/+ on C57BL/6 background) controls (P<0.001, Mann-Whitney U test) (FIG. 2). The addition of rat anti-mouse CXCR3 mAb 4C4 (mAb) to MLR cultures, but not an IgM control antibody (IgM), significantly reduced the MLR of wild type (WT, CXCR3+/+ on C57BL/6 background) cells stimulated with allogeneic splenocytes isolated from BALB/c mice (FIG. 3), but had no effect on mitogen-induced responses.

Disruption of CXCR3 provided no benefit in two classical models of T-cell dependent pathology (Toxoplasmosis gondii infection, lymphochoriomeningitis infection).

### Example 5

### Administering anti-CXCR3 mAb 4C4 to Recipient Mice Prolonged the Survival of Cardiac Allografts

Methods

Mouse cardiac allografting. Cardiac allografts derived from BALB/c donors were transplanted into wild type (WT, CXCR3+/+ on C57BL/6 background) mice as described in Example 1.

Therapeutic Intervention. CXCR3+/+ allograft recipients were administered rat anti-mouse CXCR3 monoclonal antibody 4C4 (mAb) or IgM control antibody (IgM). Antibodies (500 μg) were administered to recipient mice by intraperitoneal injection. A first dose of antibody was administered at the time of transplantation (day 0, d 0) or 4 days after transplantation (day 4, d +4). Additional doses were administered every 48 hours until day 14.

Results

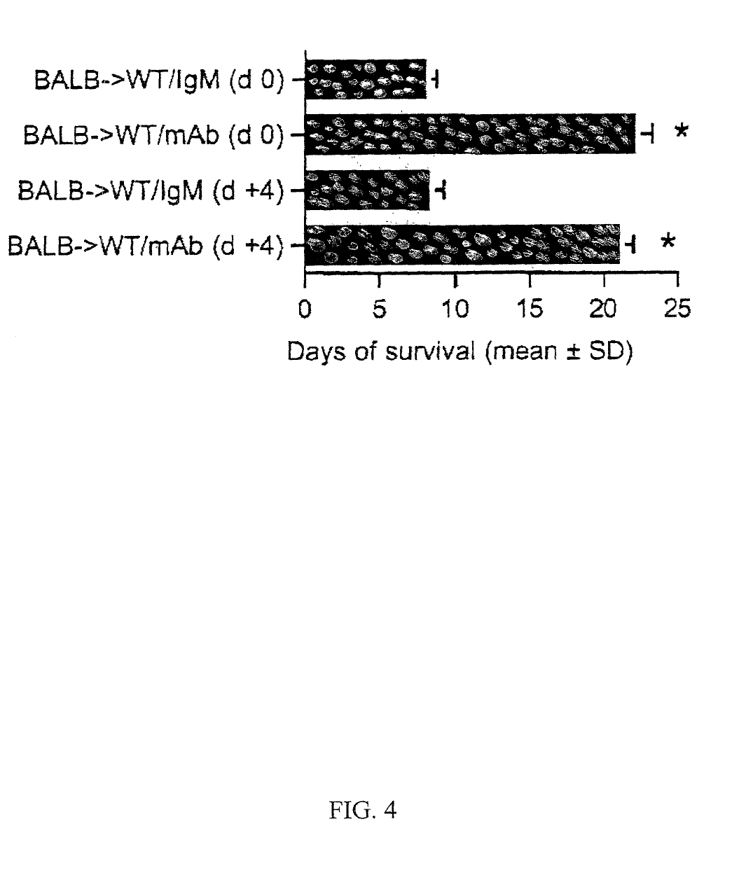

Administration of rat anti-mouse CXCR3 monoclonal antibody 4C4 (mAb) to CXCR3+/+ mice significantly prolonged allograft survival (p<0.001, Mann-Whitney U test) (FIG. 4). Administration of anti-CXCR3 mAb 4C4 (mAb) (every 48 hours until day 14) significantly prolonged the survival of allogeneic cardiac allografts when administration began at transplantation (day 0, d 0) (p<0.001, as compared with recipients that were administered an IgM control antibody (IgM)). The survival of allogeneic cardiac allografts was also prolonged when anti-CXCR3 mAb 4C4 (mAb) administration (every 48 hours until day 14) was initiated four days after transplantation when graft rejection had begun (day 4, d +4) (p<0.001, as compared with recipients that were administered an IgM control antibody (IgM)).

These results demonstrate that disruption of CXCR3 function dramatically inhibited allograft rejection. The results further demonstrate that agents (e.g., anti-CXCR3 antibodies) which inhibit CXCR3 function can be administered after rejection has commenced to inhibit (e.g., reduce, prevent) further rejection and/or stop rejection.

While this invention has been particularly shown and described with references to preferred embodiments thereof, it will be understood by those skilled in the art that various changes in form and details can be made therein without departing from the spirit and scope of the invention as defined by the appended claims.

What is claimed is:

1. A method of inhibiting graft rejection comprising administering to a subject in need thereof an effective amount of an antagonist of CXCR3 function.

2. The method of claim 1, wherein said graft is an allograft.

3. The method of claim 2, wherein said allograft is selected from the group consisting of kidney, liver, lung, heart-lung, pancreas, bowel and heart.

4. The method of claim 3, wherein said allograft is a heart.

5. The method of claim 1, wherein said antagonist of CXCR3 function is selected from the group consisting of small organic molecules, natural products, peptides, proteins and peptidomimetics.

6. The method of claim 5, wherein said antagonist of CXCR3 function is a small organic molecule.

7. The method of claim 5, wherein said antagonist of CXCR3 function is a natural product.

8. The method of claim 5, wherein said antagonist of CXCR3 function is a peptide.

9. The method of claim 5, wherein said antagonist of CXCR3 function is a peptidomimetic.

10. The method of claim 5, wherein said antagonist of CXCR3 function is a protein.

11. The method of claim 10, wherein said protein is an anti-CXCR3 antibody or antigen-binding fragment thereof.

12. The method of claim 1, wherein the graft has reduced capacity to express a ligand for CXCR3.

13. A method of inhibiting graft rejection comprising administering to a subject in need thereof an effective amount of an antagonist of CXCR3 function and an effective amount of an immunosuppressive agent.

14. The method of claim 13, wherein said immunosuppressive agent is one or more agents selected from the group consisting of calcineurin inhibitors, glucocorticoids, nucleic acid synthesis inhibitors, and antibodies which bind to lymphocytes or antigen-binding fragments thereof.

15. The method of claim 14, wherein said immunosuppressive agent is a calcineurin inhibitor.

16. The method of claim 15, wherein said calcineurin inhibitor is cyclosporin A.

17. The method of claim 15, wherein said calcineurin inhibitor is FK-506.

18. The method of claim 14, wherein said immunosuppressive agent is a glucocorticoid.

19. The method of claim 18, wherein said glucocorticoid is prednisone or methylprednisolone.

20. A method of inhibiting graft versus host disease comprising administering an effective amount of an antagonist of CXCR3 function to a recipient of a transplanted graft.

21. The method of claim 20, wherein said graft is bone marrow.

22. The method of claim 21, further comprising administering an immunosuppressive agent.

23. The method of claim 22, wherein said immunosuppressive agent is a calcineurin inhibitor.

24. The method of claim 23, wherein said calcineurin inhibitor is cyclosporin A or FK-506.

25. A method for diagnosing graft rejection comprising assessing the expression of a ligand of CXCR3 by said graft, wherein the expression of said ligand or elevated expression of said ligand relative to a suitable control is indicative of graft rejection.

26. The method of claim 25, wherein said ligand is IP-10.

27. The method of claim 25, wherein the expression of said ligand is assessed in a sample of said graft obtained by biopsy.

28. The method of claim 26, wherein said graft is a cardiac graft.

29. A method for diagnosing graft rejection comprising assessing the expression of a ligand of CCR1 by said graft, wherein the expression of said ligand or elevated expression of said ligand relative to a suitable control is indicative of graft rejection.

30. The method of claim 29, wherein said ligand is RANTES.

31. The method of claim 29, wherein the expression of said ligand is assessed in a sample of said graft obtained by biopsy.

32. The method of claim 31, wherein said graft is a cardiac graft.

33. A method for diagnosing graft rejection comprising assessing graft infiltration by CXCR3⁺ cells and/or CCR1⁺ cells, wherein the presence of CXCR3⁺ cells and/or CCR1⁺ cells in the graft or the presence of elevated numbers of CXCR3⁺ cells and/or CCR1⁺ cells in the graft relative to a suitable control is indicative of graft rejection.

34. The method of claim 33, wherein graft infiltration by CXCR3⁺ cells is assessed in a sample of said graft obtained by biopsy.

35. The method of claim 34, wherein said graft is a cardiac graft.

36. The method of claim 33, wherein graft infiltration by CCR1⁺ cells is assessed in a sample of said graft obtained by biopsy.

37. The method of claim 34, wherein said graft is a cardiac graft.

38. A method of inhibiting chronic rejection of a transplanted graft comprising administering to a subject in need thereof an effective amount of an antagonist of CXCR3 function.

39. The method of claim 38, wherein said graft is an allograft.

40. The method of claim 39, wherein said allograft is selected from the group consisting of kidney, liver, lung, heart-lung, pancreas, bowel and heart.

41. The method of claim 40, wherein said allograft is a heart.

42. The method of claim 38, wherein said antagonist is an antibody or antigen-binding fragment thereof which binds CXCR3.

43. The method of claim 42, wherein said antibody or antigen-binding fragment thereof binds CXCR3 and inhibits the binding of a ligand to CXCR3.

44. The method of claim 38, further comprising administering to said subject an effective amount of an immunosuppressive agent.

45. The method of claim 44, wherein said immunosuppressive agent is one or more agents selected from the group consisting of calcineurin inhibitors, glucocorticoids, nucleic acid synthesis inhibitors and antibodies which bind to lymphocytes or antigen-binding fragments thereof.

46. The method of claim 45, wherein said immunosuppressive agent is a calcineurin inhibitor.

47. The method of claim 46, wherein said calcineurin inhibitor is cyclosporin A.

48. The method of claim 46, wherein said calcineurin inhibitor is FK-506.

49. The method of claim 45, wherein said immunosuppressive agent is a glucocorticoid.

50. The method of claim 49, wherein said glucocorticoid is prednisone or methylprednisolone.

51. A method of treating a subject who is rejecting a graft comprising administering to said subject an effective amount of an antagonist of CXCR3 function, whereby graft rejection is inhibited.

52. The method of claim 51, wherein said graft is an allograft.

53. The method of claim 52, wherein said allograft is selected from the group consisting of kidney, liver, lung, heart-lung, pancreas, bowel and heart.

54. The method of claim 53, wherein said allograft is a heart.

55. The method of claim 51, wherein said antagonist is an antibody or antigen-binding fragment thereof which binds CXCR3.

56. The method of claim 55, wherein said antibody or antigen-binding fragment thereof binds CXCR3 and inhibits the binding of a ligand to CXCR3.

57. The method of claim 51, further comprising administering to said subject an effective amount of an immunosuppressive agent.

58. The method of claim 57, wherein said immunosuppressive agent is one or more agents selected from the group consisting of calcineurin inhibitors, glucocorticoids, nucleic acid synthesis inhibitors and antibodies which bind to lymphocytes or antigen-binding fragments thereof.

59. The method of claim 58, wherein said immunosuppressive agent is a calcineurin inhibitor.

60. The method of claim 59, wherein said calcineurin inhibitor is cyclosporin A.

61. The method of claim 59, wherein said calcineurin inhibitor is FK-506.

62. The method of claim 58, wherein said immunosuppressive agent is a glucocorticoid.

63. The method of claim 62, wherein said glucocorticoid is prednisone or methylprednisolone.

64. A method of inhibiting graft rejection comprising administering to a subject in need thereof an effective amount of an antibody or antigen-binding fragment thereof which binds IP-10 and inhibits the binding of IP-10 to CXCR3.

65. The method of claim 64, wherein said graft is an allograft.

66. The method of claim 65, wherein said allograft is selected from the group consisting of kidney, liver, lung, heart-lung, pancreas, bowel and heart.

67. The method of claim 66, wherein said allograft is a heart.

68. The method of claim 64, further comprising administering to said subject an effective amount of an immunosuppressive agent.

69. The method of claim 68, wherein said immunosuppressive agent is one or more agents selected from the group consisting of calcineurin inhibitors, glucocorticoids, nucleic acid synthesis inhibitors and antibodies which bind to lymphocytes or antigen-binding fragments thereof.

70. The method of claim 69, wherein said immunosuppressive agent is a calcineurin inhibitor.

71. The method of claim 70, wherein said calcineurin inhibitor is cyclosporin A.

72. The method of claim 70, wherein said calcineurin inhibitor is FK-506.

73. The method of claim 69, wherein said immunosuppressive agent is a glucocorticoid.

74. The method of claim 73, wherein said glucocorticoid is prednisone or methylprednisolone.

75. A method of inhibiting graft rejection comprising administering to a subject in need thereof an effective amount of an antibody or antigen-binding fragment thereof which binds Mig and inhibits the binding of Mig to CXCR3, wherein said graft is selected from the group consisting of kidney, liver, lung, heart-lung, pancreas, bowel and heart.

76. The method of claim 75, wherein said graft is a heart.

77. The method of claim 75, further comprising administering to said subject an effective amount of an immunosuppressive agent.

78. The method of claim 77, wherein said immunosuppressive agent is one or more agents selected from the group consisting of calcineurin inhibitors, glucocorticoids, nucleic acid synthesis inhibitors and antibodies which bind to lymphocytes or antigen-binding fragments thereof.

79. The method of claim 78, wherein said immunosuppressive agent is a calcineurin inhibitor.

80. The method of claim 79, wherein said calcineurin inhibitor is cyclosporin A.

81. The method of claim 79, wherein said calcineurin inhibitor is FK-506.

82. The method of claim 78, wherein said immunosuppressive agent is a glucocorticoid.

83. The method of claim 82, wherein said glucocorticoid is prednisone or methylprednisolone.

84. A method of inhibiting graft rejection comprising administering to a subject in need thereof an effective amount of an antibody or antigen-binding fragment thereof which binds I-TAC and inhibits the binding of I-TAC to CXCR3.
